(19) United States
(12) Patent Application Publication
(10) Pub. No.: US 2011/0259084 A1
(43) Pub. Date: Oct. 27, 2011

(54) GAS SENSOR UNIT

(75) Inventors: Takayoshi ATSUMI, Konan-shi (JP); Yasuhiro FUJITA, Kaizu-shi (JP)

(73) Assignee: NGK SPARK PLUG CO., LTD., Nagoya-shi (JP)

(21) Appl. No.: 13/091,281

(22) Filed: Apr. 21, 2011

(30) Foreign Application Priority Data
Apr. 23, 2010 (JP) 2010-099493
Dec. 24, 2010 (JP) 2010-286994

(51) Int. Cl. G01N 33/00

(52) U.S. Cl. 73/31.05

(57) ABSTRACT

A gas sensor unit including: a gas sensor which includes a gas detection element, a sensor terminal and a cylindrical surrounding body which surrounds a periphery of the rear end of the sensor terminal; and a sensor cap. The sensor cap includes a cap terminal and a cap body which has an insertion hole surrounding a part of the rear end of the surrounding body. The cap body is formed of a resin, and a cap body seal surrounding the surrounding body is fitted into the insertion hole. The cap body includes a rear end seal pressing portion that contacts a rear end side surface of the cap body seal; and a leading end seal pressing portion that contacts a leading end side surface of the cap body seal, which holds the cap body seal.

### BACKGROUND OF THE INVENTION

1. Field of the Invention

The present invention relates to a gas sensor unit that comprises a gas sensor having a gas detection element and a sensor cap mounted on the gas sensor, the gas sensor unit transmitting an output of the gas sensor to an external device.

2. Description of the Related Art

There are various types of gas sensors having a gas detection element. As one of gas sensors, for example, a gas sensor may be exemplified which has a gas detection element formed of a solid-state electrolyte body having oxygen ion conductivity and which is attached to an attachment object such as an exhaust pipe or an engine head of an internal combustion engine so as to detect a concentration of a specific gas (for example, oxygen) contained in an exhaust gas. Further, in the gas sensor, a sensor cap is detachably attached to the rear end of the gas sensor in order to transmit the output from the gas sensor to an external device, thereby forming the gas sensor unit (for example, refer to Patent Documents 1 and 2).

As shown in FIG. 10, a related art gas sensor unit 900 includes a gas sensor 1 and a sensor cap 100. The gas sensor 1 and the sensor cap 100 are attachable to or detachable from each other, and are relatively rotatable in the circumferential direction of the gas sensor 1.

The gas sensor 1 includes a gas detection element 2 which extends in the axial direction and is formed as a bottomed cylinder having a leading end exposed to a detection gas, and a metal shell 3 which surrounds the periphery of the gas detection element 2. The gas detection element 2 includes an outer electrode 4 and an inner electrode 5 which are respectively provided at the outer surface and the inner surface thereof. A cylindrical terminal member 6 is inserted into a round hole of the rear end side of the gas detection element 2 so as to be connected to the inner electrode 5 and so as to transmit the output signal from the gas detection element 2 to the external device. Further, the rear end side of the metal shell 3 is provided with a surrounding body 7 which surrounds the rear end side of the gas detection element 2 and the terminal member 6 and is held by the metal shell 3.

On the other hand, the sensor cap 100 includes a cap terminal 101 which is substantially formed in a double cylindrical shape and a cylindrical terminal surrounding portion 102 which is formed of an elastic member (for example, a rubber member) and which surrounds the rear end side of the surrounding body 7 and the cap terminal 101. The terminal surrounding portion 102 is provided with an insertion hole 110 into which the surrounding body 7 is inserted. The cap terminal 101 includes a cylindrical portion 101a which extends from the center in the axial direction and has a bottomed leading end and a flange surface which is widened outward in the radial direction in a concentric shape from the rear edge of the cylindrical portion 101a, where the outer peripheral edge of the flange surface protrudes downward to the leading end side. Then, when the cylindrical portion 101a is inserted into the terminal member 6 of the gas sensor 1, both are electrically connected to each other so that the output signal is transmitted to the external device. Further, the sensor cap 100 includes a lead wire surrounding portion 104 which protrudes outward in the radial direction from the terminal surrounding portion 102 and covers the lead wire 103 connected to the cap terminal 101. Accordingly, the lead wire 103 is drawn outward in the radial direction from the sensor cap 100 (the terminal surrounding portion 102 and the lead wire surrounding portion 104). Further, the sensor cap 100 includes a filter surrounding portion 105 which protrudes outward in the radial direction of the terminal surrounding portion 102 and has a communication hole 111 allowing communication between the inside and the outside of the terminal surrounding portion 102. A filter member 106 having air permeability and hydrophobic properties is disposed inside the filter surrounding portion 105 so as to block the communication hole 111.

Further, since the terminal surrounding portion 102 is formed of a rubber member, a gap between the surrounding body 7 and the terminal surrounding portion 102 is water-tightly sealed, thereby preventing a short-circuit caused by the intrusion of a foreign matter such as water into the gas sensor 1.

[Patent Document 1] JP-A-2007-107935

[Patent Document 2] JP-A-2008-256544

3. Problem to be Solved by the Invention

However, in the case of the related art gas sensor unit 900, the entire sensor cap 100 is formed of a rubber member from the viewpoints of convenience in attachment/detachment of the gas sensor 1 and forming a seal between the gas sensor 1 and the bonding portion. However, since the rubber member capable of sealing the bonding portion and having elasticity is expensive, the manufacturing cost increases. Further, since thermal degradation occurs in the sensor cap 100 formed of the rubber member due to heat transferred from the internal combustion engine or the like, the sensor cap 100 must be exchanged as needed.

### SUMMARY OF THE INVENTION

It is therefore an object of the invention to provide a gas sensor unit which is inexpensive and in which a rubber seal member is easily exchangeable.

The above object has been achieved, according to a first aspect (1) of the invention, by providing a gas sensor unit comprising: a gas sensor including a gas detection element which extends in an axial direction and has a cylinder shape with a bottom in which a leading end side is closed, a sensor terminal which is inserted into a round hole of the gas detection element to be electrically connected to an inner electrode formed at an inner surface of the gas detection element and having a rear end protruding from the rear end side of the gas detection element, and a cylindrical surrounding body which surrounds a periphery of the rear end of the sensor terminal; and a sensor cap including a cap terminal which is electrically connected to the sensor terminal, and a cap body which has an insertion hole surrounding at least a part of the rear end of the surrounding body, the cap body surrounding a periphery of the cap terminal, wherein the cap body is formed of a resin, a cap body seal surrounding the surrounding body is fitted into the insertion hole, and wherein the cap body includes a rear end seal pressing portion that contacts a rear end side surface of the cap body seal; and a leading end seal pressing portion that contacts a lending end surface of the cap body seal, wherein the cap body seal is held by the rear end seal pressing portion and the leading end pressing portion of the cap body.

With such a configuration, the cap body is formed of a resin, and the cap body seal held between the rear end seal pressing portion and the leading end seal pressing portion of the cap body may be formed of an elastic member such as rubber. Accordingly, the product cost may be reduced as compared with the case where the entire cap body is formed of a rubber member, or where a large rubber member is disposed to entirely cover a gap between the cap body and the gas sensor.

As one method of fixing the rubber seal member to the bonding portion, a method of fixing the rubber seal member to the bonding portion by ultrasonic welding may be exemplified. However, in ultrasonic welding, the number of manufacturing steps increases, the welded rubber seal member is difficult to exchange, and the rubber seal member may not be exchangeable in the event of thermal degradation.

As another method of reliably fixing the rubber seal member to the bonding portion, a method may be exemplified which allows a large rubber member entirely covering the outside of the gas sensor 1 and the sensor cap 100 to be fitted to the outside of the gas sensor unit 900. However, when a large rubber seal member is used, the volume of the rubber member increases so that the manufacturing cost increases. For this reason, such configuration is not desirable.

To the contrary, in the invention, the leading end side and the rear end side of the cap body seal are held by the rear end seal pressing portion and the leading end seal pressing portion of the cap body, respectively, so as to reliably hold the cap body seal.

Further, even when thermal degradation occurs in the cap body seal under practical use, the cap body seal may be easily exchanged in accordance with the invention.

The “resin” used herein does not include an elastic material (elastic rubber) such as natural rubber or synthetic rubber.

The insertion hole is formed to surround at least a part of the rear end of the surrounding body, but more desirably, the insertion hole is formed to surround the entire rear end of the surrounding body.

In a preferred embodiment (2) of the gas sensor unit (1), the cap body includes a main cap body portion having the rear end seal pressing portion and a seal stopper fitting into the insertion hole and having the leading end seal pressing portion, the main cap body portion and the seal stopper being provided as separate components. Further, the seal stopper is coupled to the main cap body portion at a leading end side of the cap body seal so as to hold the cap body seal.

With such a configuration, since the rear end seal pressing portion and the leading end seal pressing portion are provided as separate components, and the cap body seal is held therebetween, the productivity is enhanced.

In another preferred embodiment (3) of the gas sensor unit (2), a concave portion or an open portion is provided at a leading end side of the main cap body portion. The seal stopper has an outward engagement portion protruding outward in a radial direction to engage the concave portion or the open portion, and the seal stopper is fitted to the inside of the main cap body portion.

With such a configuration, since a clicking sensation is generated when the concave portion or the open portion engages the engagement portion, a sensation of mounting the seal stopper is obtained. For this reason, since it is easy to know whether the seal stopper and the main cap body are coupled to each other in the manufacturing process, the seal stopper may be reliably coupled to the main cap body portion. Further, when an open portion is provided in the main cap body portion instead of a concave portion, since the main cap body portion and the seal stopper may be separated from each other by pressing the outward engagement portion from the outside of the cap body via the open portion so that the seal stopper is deformed, the cap body seal may be easily exchanged. For this reason, this configuration is more desirable.

In yet another preferred embodiment (4) of the gas sensor unit (2), an outer surface of the main cap body portion has a concave portion or an open portion. The seal stopper has an inward engagement portion protruding inward in the radial direction to engage with the concave portion or the open portion, and the seal stopper is fitted to the outside of the main cap body portion.

With such a configuration, since a clicking sensation is generated when the concave portion or the open portion engages the engagement portion, a sensation of mounting the seal stopper is obtained. For this reason, since it is easy to know whether the seal stopper and the main cap body are coupled to each other in the manufacturing process, the seal stopper may be reliably coupled to the main cap body portion. Further, since the main cap body portion and the seal stopper may be separated from each other by pulling the inward engagement portion outward in the radial direction, the cap body seal may be easily exchanged.

In yet another preferred embodiment (5), the gas sensor unit (1) further comprises a metal shell which supports the gas detection element; and a metallic outer cylinder which surrounds the outside of the surrounding body, wherein the outer cylinder is fixed to the metal shell.

Since the surrounding body surrounds the periphery of the sensor terminal, the surrounding body is formed of a non-conductive material (resin, ceramic, or the like) other than metal or a material obtained by performing an insulation process on the surface of the metal. For this reason, it is difficult to directly fix the metallic metal shell and the surrounding body to each other. However, with such a configuration, the surrounding body may be easily fixed to the metal shell via the outer cylinder.

In yet another preferred embodiment (6) of the gas sensor unit (5), the outer cylinder is crimped and fixed to the metal shell.

With such a configuration, since the outer cylinder and the surrounding body may be easily fixed to the metal shell, the productivity is enhanced.

In yet another preferred embodiment (7) of the gas sensor unit (1), at least a part of the cap body seal is located at a rear end side of the sensor cap rather than at the center thereof when viewed from the axial direction.

Thermal degradation easily occurs in the cap body seal due to heat transferred from an attachment object such as an exhaust pipe or an engine head of an internal combustion engine to which the gas sensor unit is attached. However, according to the configuration of the invention, since the distance from the attachment object to the cap body seal becomes more distant relative to each other, thermal degradation of the cap body seal may be effectively prevented.

According to the invention, a gas sensor unit capable of holding a seal at a bonding portion between the gas sensor and the sensor cap may be obtained at a low cost.

### BRIEF DESCRIPTION OF THE DRAWINGS

Illustrative aspects of the invention will be described in detail with reference to the drawings wherein:

FIG. 1 is a perspective view illustrating a gas sensor unit according to a first embodiment;

FIG. 2 is a cross-sectional view taken along the line II-II of FIG. 1;

FIG. 3 is a partial cross-sectional view illustrating a gas sensor and a sensor cap;

FIG. 4 is a cross-sectional view illustrating a state where a main cap body portion and a seal stopper are assembled;

FIG. 5 is a perspective view illustrating a state where the main cap body portion and a cover portion are assembled;

FIG. 6 is a perspective view illustrating a structure of the cover portion at its leading end surface;

FIG. 7 is a cross-sectional view taken along the line C-C of FIG. 5;

FIG. 8 is a partial cross-sectional view illustrating a gas sensor unit according to a second embodiment;

FIG. 9 is a partial cross-sectional view illustrating a gas sensor unit according to a third embodiment; and

FIG. 10 is a cross-sectional view illustrating a gas sensor unit according to the related art.

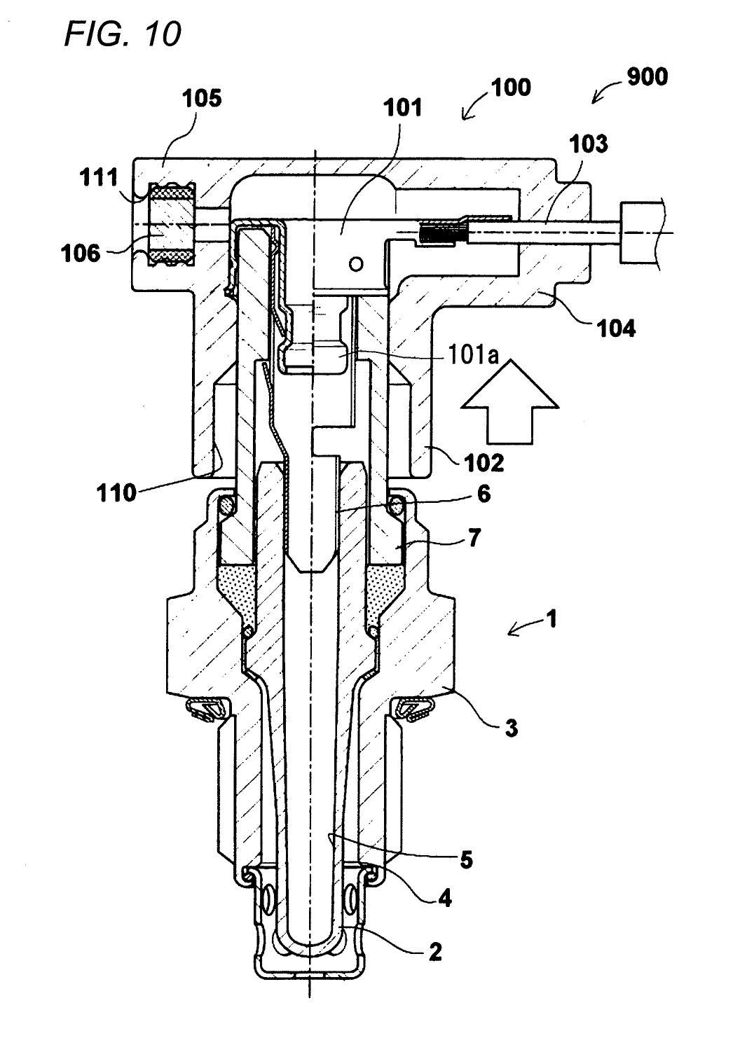

### DETAILED DESCRIPTION OF THE PREFERRED EMBODIMENTS

Hereinafter, various embodiments of the invention will be described in detail. However, the present invention should not be construed as being limited thereto.

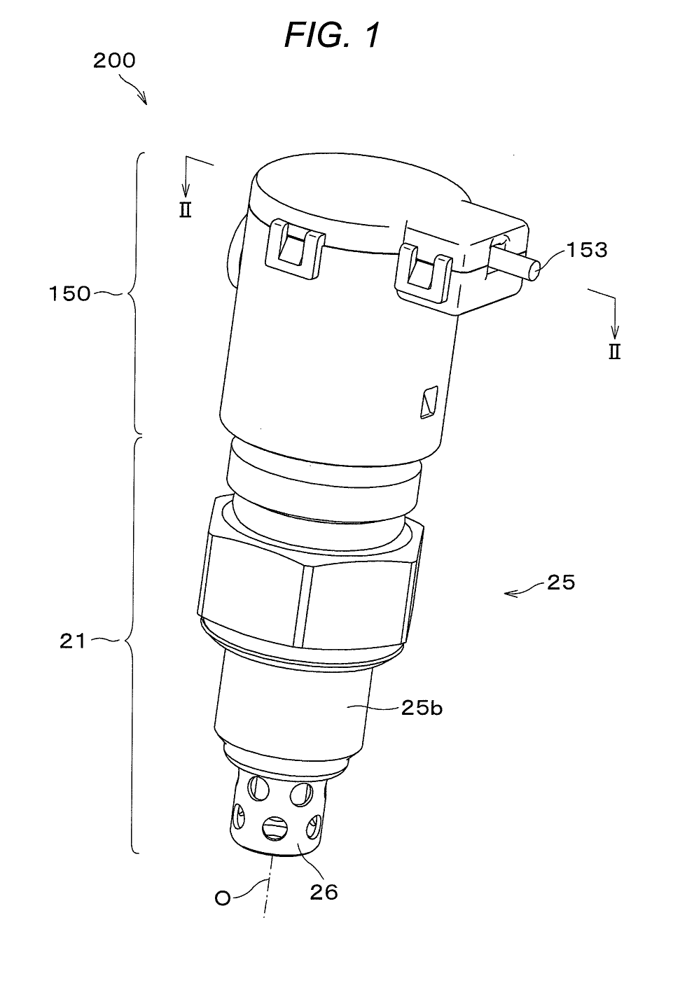

FIG. 1 is a perspective view illustrating a gas sensor unit 200 according to a first embodiment. The gas sensor unit 200 includes a gas sensor 21 and a sensor cap 150 which is disposed at the rear end side (the upper side of FIG. 1) of the gas sensor 21 in the axial direction O. The gas sensor 21 is an oxygen sensor that includes a metal shell 25 and which measures the concentration of oxygen contained in exhaust gas. The gas sensor 21 is put to use by fixing a screw portion 25b located on the leading end side of the metal shell 25 to an attachment object such as an exhaust pipe or an engine head by a screw, and projecting the leading end of the gas sensor 21 toward the attachment object. Further, a protector 26 is attached to the leading end of the gas sensor 21. On the other hand, the sensor cap 150 is substantially formed in a cylindrical shape, and a lead wire 153 is drawn from the side portion of the rear end side of the sensor cap 150 to the outside thereof.

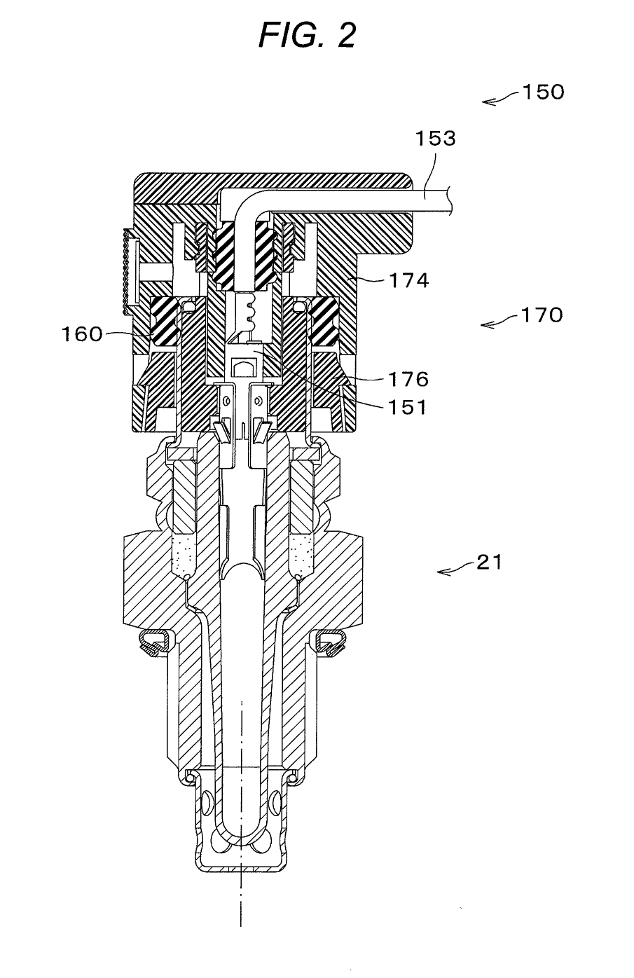

FIG. 2 is a cross-sectional view taken along the line II-II of FIG. 1. The gas sensor 21 and the sensor cap 150 are coupled to each other so as to be attachable to and detachable from each other and to be rotatable relative to each other in the circumferential direction. The sensor cap 150 includes a cap terminal 151, a cap body 170 surrounding the periphery of the cap terminal 151, and the lead wire 153 electrically connected to the rear end side of the cap terminal 151. Further, the cap body 170 includes a main cap body portion 174 and a seal stopper 176, and a rubber-like cap body seal 160 is interposed therebetween.

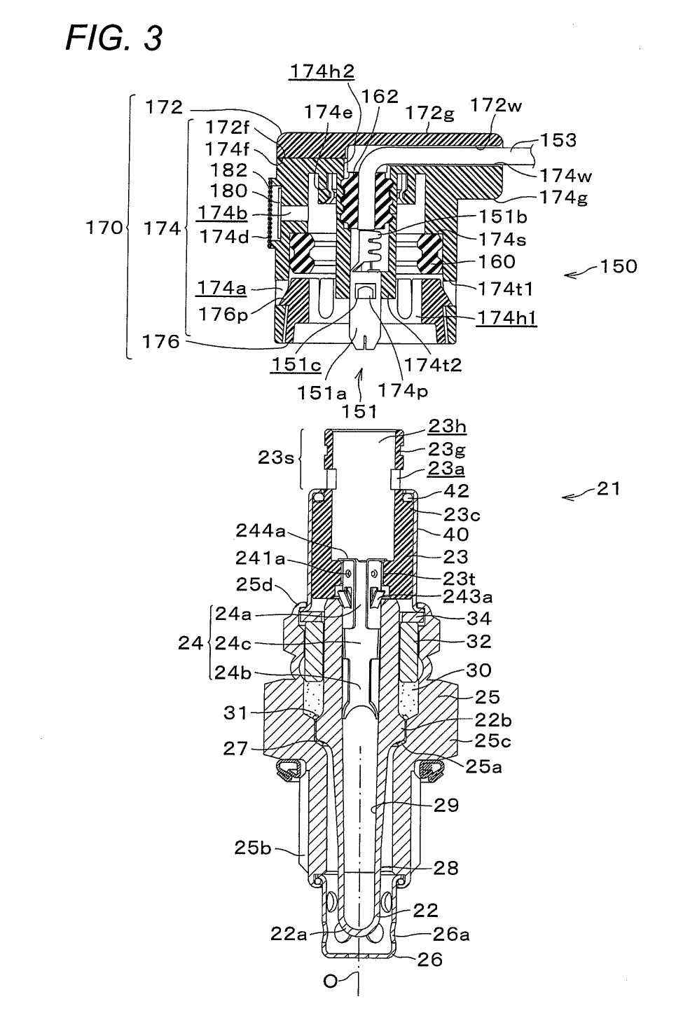

Hereinafter, the configuration of the gas sensor 21 and the sensor cap 150 will be described in more detail by reference to FIG. 3 showing a state where the gas sensor 21 and the sensor cap 150 are detached from each other.

As shown in FIG. 3, the gas sensor 21 includes a gas detection element 22, a resinous surrounding body 23, a sensor terminal 24, a metallic outer cylinder 40, and the metal shell 25.

Further, in the description below, the attachment side of the sensor cap 150 in the direction along the axis O will be described as the rear end side, and the opposite side thereof will be described as the leading end side.

The metal shell 25 is made of stainless steel, and is formed in a cylindrical shape. The metal shell 25 is provided with an inner periphery receiving portion 25a that supports a flange portion 22b of a gas detection element 22 described below. The inner periphery receiving portion 25a decreases in diameter toward the leading end side (the lower side in FIG. 3), and is provided in the circumferential direction so as to protrude inward in the radial direction from the inner peripheral surface of the metal shell 25. Further, the screw portion 25b is formed at the outside of the metal shell 25 to attach the gas sensor 21 to the attachment object, and a hexagonal portion 25c is circumferentially provided at the rear end side (the upper side in FIG. 3) of the screw portion 25b to engage with an attachment tool that is used to allow the screw portion 25b to be threaded into the attachment object. A protector 26 is attached to the leading end side of the metal shell 25 to cover a leading end portion 22a of the gas detection element 22 described below. The protector 26 is made of metal to be formed as a cylindrical bottomed cylinder, and includes plural air passage holes 26a that are used to introduce exhaust gas of the attachment object into the gas sensor 21.

The gas detection element 22 is formed of a solid-state electrolyte body having oxygen ion conductivity, and is formed in a bottomed cylindrical shape of which the leading end portion 22a is closed and which extends in the axial direction O. The flange portion 22b is provided at the outer periphery of the gas detection element 22 to protrude outward in the radial direction, and the gas detection element 22 is disposed inside the metal shell 25 while a metallic packing 27 is interposed between the leading end side surface of the flange portion 22b and the surface of the inner periphery receiving portion 25a of the metal shell 25. Further, as a solid electrolyte body forming the gas detection element 22, for example, ZrO₂ formed as a solid solution with CaO or Y₂O₃ is a representative example thereof, but a solid solution of ZrO₂ and an oxide of rare-earth metal or alkali earth metal may be used. In addition, HfO₂ may be contained therein.

An outer electrode 28 is formed in the outer peripheral surface of the leading end portion 22a of the gas detection element 22. The outer electrode 28 is formed of Pt or a Pt alloy and so as to be porous. The outer electrode 28 is provided up to the leading end side surface of the flange portion 22b, and is electrically connected to the metal shell 25 via the metallic packing 27. For this reason, the potential of the outer electrode 28 may be taken as that of the metal shell 25.

On the other hand, an inner electrode 29 is formed in the inner peripheral surface of the gas detection element 22. The inner electrode 29 is also formed of Pt or a Pt alloy and so as to be porous.

The annular packing 31 is disposed at the rear end side of the flange portion 22b of the gas detection element 22, and a gap between the gas detection element 22 and the metal shell 25 closer to the rear end side than the annular packing 31 is filled with a ceramic powder 30. Further, an annular ceramic ring 32 and an annular metallic ring 34 are laminated in this order in a gap between the gas detection element 22 and the metal shell 25 closer to the rear end side than the ceramic powder 30, and are crimped to an outer cylinder 40 to be described below by a crimping portion 25d located at the rear edge of the metal shell 25. The outer cylinder 40 is integrally fixed to the metal shell 25, and presses the ceramic powder 30 toward the leading end side via the ceramic ring 32 by action of the crimping portion 25d. Further, the ceramic powder 30 may be formed of a known seal material such as talc used in the gas sensor.

The surrounding body 23 is formed of a known resin such as PBT (poly butylene terephthalate) or PPS (polyphenylene sulfide), and is formed in a double cylindrical shape that includes a rear end 23s decreasing in diameter toward the rear end side and a surrounding body leading end 23c having a diameter larger than that of the rear end 23s. In addition, the outer peripheral surface at the rear end side of the surrounding body leading end 23c is notched, and an O-ring 42 described below is fitted to the outside thereof. Further, a flange portion 23t is formed at the leading end side of an inner hole 23h of the surrounding body 23 to protrude inward in the radial direction. The inner diameter of the flange portion 23t is substantially equal to the outer diameter of a sensor terminal 24 described below. On the other hand, a groove (concave portion) 23g is formed in the outer surface of the rear end 23s to be continuous in the circumferential direction.

Further, plural air passage holes 23a are intermittently formed in the circumferential direction between the groove 23g and the surrounding body leading end 23c in the outer surface of the rear end 23s, and allow for introduction of a reference gas into the air passage holes of the gas detection element 22 via a communication hole 174b of the sensor cap 150 described below.

The metallic outer cylinder 40 is formed in a cylindrical shape of which the inner diameter is slightly larger than the outer diameter of the surrounding body leading end 23c, the rear edge extends inward in the radial direction to be formed as an annular rear end surface, and the front edge increases in diameter to be formed as a flange. Then, when the surrounding body 23 having the O-ring 42 mounted thereon is received in the outer cylinder 40, the rear end 23s of the surrounding body 23 protrudes from the central hole of the rear end surface of the outer cylinder 40. In this state, the front edge of the outer cylinder 40 is crimped by the crimping portion 25d, the rear end surface of the outer cylinder 40 biases the O-ring 42 toward the leading end side, and the leading end of the surrounding body leading end 23c comes into contact with the rear end side surface of the gas detection element 22 in accordance with the pressing action of the O-ring 42. That is, instead of the resinous surrounding body 23 is directly crimped by the crimping portion 25d, the surrounding body 23 is fixed to the metal shell 25 by the elastic force of the O-ring 42.

Although it is difficult to directly fix the metallic metal shell 25 and the resinous surrounding body 23 to each other, in this configuration, the resinous surrounding body 23 may be reliably fixed to the metal shell 25 via the outer cylinder 40.

The sensor terminal 24 is made of, for example, INCONEL (trademark of Special Metals Corp., N.Y.) to be formed in a cylindrical shape, and includes an output side terminal portion 24a, an element side terminal portion 24b, and a terminal connection portion 24c connecting both portions to each other.

Among these components, the output side terminal portion 24a is formed in a cylindrical shape of which a cross-section perpendicular to the axis O is substantially a C-shape. Then, when the cap terminal 151 described below is moved relative to the gas sensor 21 in a direction along the axis O (the vertical direction of FIG. 3) to be inserted into the output side terminal portion 24a, the output side terminal portion 24a elastically increases in diameter. Further, a convex portion 241a is formed at four positions in the circumferential direction at the rear end side (the upper side of FIG. 3) of the output side terminal portion 24a to protrude inward in the radial direction.

Further, in the output side terminal portion 24a, an outward bent portion (separation prevention portion) 243a is formed at three positions in the circumferential direction closer to the leading end side (the lower side of FIG. 3) than the convex portion 241a so as to face the leading end side surface of the flange portion 23t of the surrounding body 23, where the outward bent portion is formed by perforating a part of the output side terminal portion 24a to be bent outward in the radial direction. At the time when the outward bent portion 243a is assembled to the sensor terminal 24 of the gas sensor 21, the outward bent portion 243a does not come into contact with the leading end side surface (step surface) of the flange portion 23t of the surrounding body 23. However, at the time when the sensor terminal 24 moves to the rear end side of the axial direction, the separation prevention portion 243a comes into contact with the leading end side surface of the flange portion 23t, to thereby prevent the sensor terminal 24 from moving any further to the rear end side in the axial direction O. That is, the outward bent portion 243a prevents separation of the output side terminal portion 24a (sensor terminal 24).

On the other hand, the element side terminal portion 24b of the sensor terminal 24 is formed in a cylindrical shape of which a cross-section perpendicular to the axis O is substantially a C-shape. The element side terminal portion 24b is inserted into the gas detection element 22 while elastically decreasing in diameter, and is electrically connected to the inner electrode 29. Accordingly, the element side terminal portion 24b is electrically connected to the inner electrode 29 while pressing on the inner electrode 29 from inside the gas detection element 22 outward in the radial direction.

Since the sensor terminal 24 is integrally molded by pressing from a predetermined-shaped single metal sheet, the sensor terminal 24 may be easily manufactured at a low cost. Further, in the sensor terminal 24 of the embodiment, a metal sheet is subjected to bending, and the output side terminal portion 24a and the element side terminal portion 24b closer to the leading end side (the lower side of FIG. 3) of the axial direction O than the output side terminal portion 24a are formed in a cylindrical shape. Further, a reference gas (external air) introduced into the sensor cap 150 is guided into the round hole of the gas detection element 22.

The gas sensor 21 may be manufactured as described below.

First, the metal shell 25 and the protector 26 are integrated with each other by crimping, welding or the like. Subsequently, the gas detection element 22 provided with the outer electrode 28 and the inner electrode 29 is inserted into the metal shell 25 together with the packing 27. Subsequently, the annular packing 31 is disposed at the rear end side of the flange portion 22b of the gas detection element 22, and the gap between the metal shell 25 and the gas detection element 22 is filled with a predetermined amount of the ceramic powder 30. Subsequently, the ceramic ring 32 and the metallic ring 34 are laminated in this order in the gap at the rear end side of the ceramic powder 30. Further, the outer cylinder 40 receiving the surrounding body 23 and the O-ring 42 are loaded at the rear end of the metallic ring 34, and the outer cylinder 40 is pressurized toward the leading end side so that the leading end flange of the outer cylinder 40 is disposed inside the crimping portion 25d. Then, in this pressurized state, the crimping portion 25d of the metal shell 25 is crimped so that the above components are integrally fixed to each other.

Finally, the sensor terminal 24 is inserted into the surrounding body 23 and the gas detection element 22. Specifically, the element side terminal portion 24b of the sensor terminal 24 is inserted from the hole 23h of the surrounding body 23 into the gas detection element 22 while elastically decreasing in diameter so as to be elastically connected to the inner electrode 29. Also, the output side terminal portion 24a is pressed into the leading end side so that a fixation portion 244a formed at the rear end of the output side terminal portion 24a comes into contact with the rear end side surface of the flange portion 23t of the surrounding body 23. The fixation portion 244a extends in a petal shape from the rear end of the output side terminal portion 24a outward in the radial direction while being perpendicular to the axis O. In this manner, the output side terminal portion 24a is disposed inside the surrounding body 23.

Further, when the output side terminal portion 24a is pressed until the fixation portion 244a comes into contact with the rear end surface of the flange portion 23t of the surrounding body 23, the outward bent portion 243a bent inward in the radial direction is released and returned to face the leading end side surface (step surface) of the flange portion 23t, whereby the separation of the sensor terminal 24 may be prevented. In this manner, the gas sensor 21 is completely assembled.

Next, the sensor cap 150 of the embodiment will be described by reference to FIG. 3. The sensor cap 150 includes the cap terminal 151, the cap body 170 covering and holding the cap terminal 151, the lead wire 153, and a filter member 180. Specifically, the cap body 170 is formed by assembling a cover portion 172, the main cap body portion 174, and the seal stopper 176, and all of these are formed of a resin such as PBT.

Further, in the invention, components (in this example, a main cap body portion 174 and a seal stopper 176) constituting a rear end seal pressing portion and a leading end seal pressing portion in the cap body 170 are essentially formed of a resin, but the other component (in this example, a cover portion 172) may be formed of a member other than a resin. However, all components of the cap body 170 may be formed of a resin, and this is desirable from the viewpoint of cost or convenience in an assembling operation.

The cap terminal 151 is made of, for example, stainless steel, and is formed by punching a sheet material. The cap terminal 151 includes a sensor connection portion 151a and a core crimping portion 151b which is integrally formed at the rear end side of the sensor connection portion 151a. The sensor connection portion 151a is formed in a cylindrical shape, and two separation prevention holes 151c described below are opened from the side surface of the sensor connection portion 151a. Further, the core crimping portion 151b is crimped to the core of the lead wire 153 to be electrically connected thereto.

The main cap body portion 174 is substantially formed in a double cylindrical shape, and includes an annular upper surface 174f at the rear end side thereof. More specifically, the main cap body portion 174 is disposed to be coaxial with a main cylinder 174t1 and an inner cylinder 174t2, and a portion of the upper surface 174f connected to the inner cylinder 174t2 is opened to communicate with an inner hole 174h2 of the inner cylinder 174t2. Hereinafter, the inner hole 174h2 will be referred to as the cap terminal hole 174h2.

The inner diameter of the leading end of the cap terminal hole 174h2 is substantially equal to the outer diameter of the cap terminal 151, and the cap terminal 151 is received inside the leading end of the inner cylinder 174t2. Further, a locking portion 174p is provided on the inside of the leading end of the inner cylinder 174t2 to protrude inward in the radial direction, and when the cap terminal 151 is inserted into the inner cylinder 174t2, the locking portion 174p is fitted into the separation prevention hole 151c to prevent separation of the cap terminal 151. Further, at a position where the locking portion 174p is fitted into the separation prevention hole 151c, the sensor connection portion 151a is exposed from the inner cylinder 174t2 to the leading end side.

On the other hand, the inner surface of the main cylinder 174t1 increases in diameter at the leading end side, and a step portion (corresponding to the “rear end seal pressing portion” of the invention) 174s is formed between the rear end and the inner surface. Then, the annular cap body seal 160 inserted from the leading end side of the main cylinder 174t1 into the main cylinder 174t1 comes into contact with the step portion 174s. Further, the annular seal stopper 176 is fitted from the leading end side of the main cylinder 174t1 into the main cylinder 174t1, so that the cap body seal 160 is held between the leading end side surface of the step portion 174s and the rear end side surface of the seal stopper 176.

The inner diameter of the rear end of the main cylinder 174t1 and the inner diameter of the seal stopper 176 are slightly larger than the outer diameter of the outer cylinder 40, so that the outer cylinder 40 (and the rear end of the surrounding body 23) may be received in the main cylinder 174t1. Further, the inner diameter of the cap body seal 160 is slightly smaller than the outer diameter of the outer cylinder 40, and when the outer cylinder 40 is received in the main cylinder 174t1, the cap body seal 160 comes into close contact with the surface of the outer cylinder 40 to obtain a waterproof assembly. Two ribs are formed in the axial direction O in the inner surface of the cap body seal 160 continuous in the circumferential direction, and are made to easily come into contact with the surface of the outer cylinder 40. The cap body seal 160 is formed of an elastic member such as rubber.

The main cylinder 174t1 and the inner hole 174h1 of the seal stopper 176 correspond to the “insertion hole” of the claimed invention.

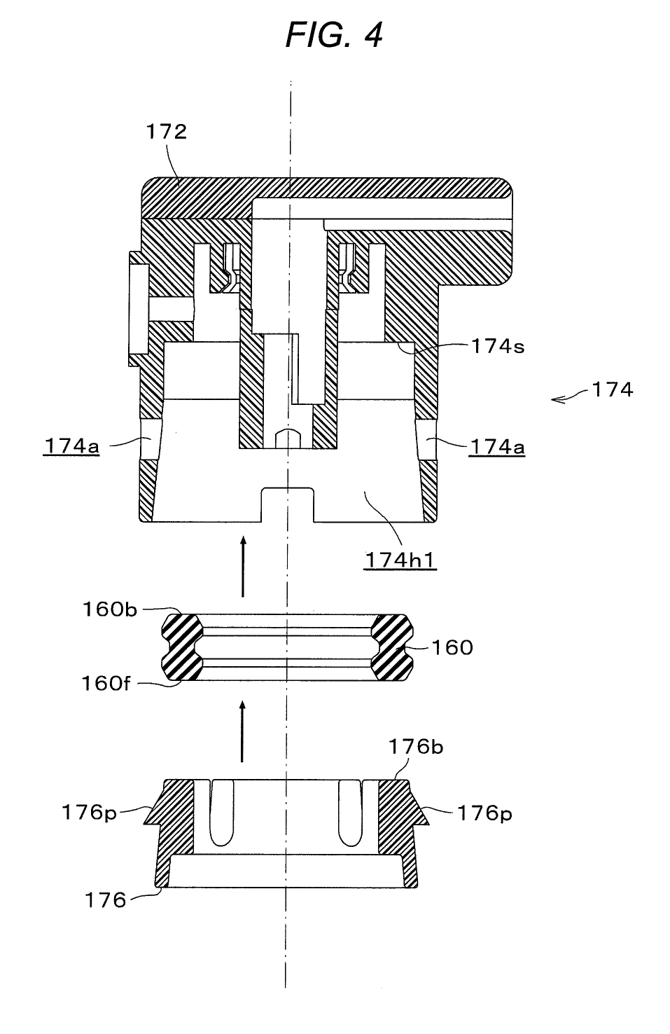

Further, as shown in FIG. 2, the rear end of the cap body seal 160 (the position corresponding to the step portion 174s of FIG. 4) is disposed closer to the rear end side than the center of the sensor cap 150 when seen from the axial direction.

Next, the assembling operation between the main cap body portion 174 and the seal stopper 176 will be described by reference to FIG. 4. As described above, the cap body seal 160 is received in the step portion 174s of the main cap body portion 174, and the seal stopper 176 is inserted into the main cap body portion 174 to hold the cap body seal 160. That is, the step portion (rear end seal pressing portion) 174s comes into contact with a rear end side surface 160b of the cap body seal 160, and an upper surface (corresponding to the “leading end seal pressing portion” of the claimed invention) 176b of the seal stopper 176 comes into contact with a leading end side surface 160f of the cap body seal 160 to hold the cap body seal 160.

Here, plural rectangular holes 174a (in this example, two facing positions in the radial direction) are opened from the side surface of the main cylinder 174t1 (refer to FIG. 3) of the main cap body portion 174. Further, plural (two in the embodiment) protrusions 176p are disposed at a position of the outer surface of the seal stopper 176 facing the rectangular holes 174a to protrude outward in the radial direction. Each rectangular hole 174a corresponds to an “open portion” of the claimed invention, and each protrusion 176p corresponds to an “engagement portion” of the claimed invention. Then, when the seal stopper 176 is inserted into the main cap body portion 174, each protrusion 176p is deformed inward in the radial direction by the elasticity of the seal stopper 176. When the seal stopper 176 faces the rectangular hole 174a, each protrusion 176p is released outward in the radial direction to be returned, whereby the protrusion engages the rectangular hole 174a so that the main cap body portion 174 and the seal stopper 176 are fixed. When each protrusion 176p engages the rectangular hole 174a, a clicking sensation is generated. For this reason, since the sensation of attaching the seal stopper 176 is obtained, the seal stopper 176 may be reliably coupled to the main cap body portion 174. Further, when the protrusion 176p is pressed via the rectangular hole 174p from the outside of the gas sensor unit 200 after the assembling operation is completed, the seal stopper 170 may be detached from the main cap body portion 174. In this manner, the cap body seal 160 may be easily exchanged after the assembling operation is completed.

Further, instead of the rectangular hole 174a, a concave portion receiving each protrusion 176p may be provided in the inner surface of the insertion hole 174h1 of the main cap body portion 174 so that the concave portion and the protrusion 176p engage each other.

The rectangular hole 174a need not be formed at two facing positions in the radial direction. That is, the rectangular holes may deviate from each other in the radial direction, or three or more rectangular holes may be provided. However, from the viewpoint of convenience in manufacturing and reliably fixing the seal stopper 176 to the main cap body portion 174, the rectangular holes 174a are desirably provided at two facing positions in the radial direction.

As described above, according to the gas sensor unit 200 of the embodiment, the cap body 170 (the main cap body portion 174 and the seal stopper 176) is formed of a resin, and the cap body seal 160 held therebetween is formed of an elastic member such as rubber. Accordingly, the product cost may be reduced compared with the case where the entire cap body 170 is formed of a rubber member, or where a large rubber member is disposed to entirely cover a gap between the cap body 170 and the gas sensor 21.

Since both the leading end side and the rear end side of the cap body seal 160 may be held by the rear end seal pressing portion and the leading end seal pressing portion, the cap body seal 160 may be reliably held.

Further, even when thermal degradation occurs in the cap body seal 160 under the practical use, the cap body seal 160 may be easily exchanged.

When the main cap body portion 174 and the seal stopper 176 are provided as separate components of the cap body 170, the cap body seal 160 may be held between the main cap body portion 174 and the seal stopper 176, whereby productivity is enhanced.

Returning to FIG. 3, in the upper surface 174f of the leading end side surface (the surface contacting the insertion hole 174h1), hook-shaped engagement portions 174e are intermittently and coaxially disposed between the main cylinder 174t1 and the inner cylinder 174t2. Further, each engagement portion 174e is formed in the shape of a piece that extends in the axial direction O, the base end is fixed to the leading end side surface of the upper surface 174f so that the engagement portion 174e is elastically bent in the radial direction, and the hook thereof faces the inside of the radial direction.

Then, when the outer cylinder 40 (and the surrounding body 23) is received in the insertion hole 174h1 of the main cylinder 174t1, the engagement portion 174e is pressed against the rear end 23s to be bent outward in the radial direction. Further, when the rear end 23s is inserted up to a position facing the groove 23g (a position where the rear edge of the surrounding body 23 substantially comes into contact with the leading end side surface of the upper surface 174f), the hook of the engagement portion 174e engages the groove 23g of the rear end 23s, and the bending of the engagement portion 174e is restored, thereby generating a clicking sensation. For this reason, since the sensation of mounting the sensor cap is obtained, the sensor cap 150 may be reliably coupled to the gas sensor 21. Further, since the groove 23g is continuous in the circumferential direction, the engagement portion 174e (and the sensor cap 150 integrated with the engagement portion 174e) may be rotated in the circumferential direction along the groove 23g. In this manner, the sensor cap 150 may be coupled to the gas sensor 21 to be rotatable in the circumferential direction.

In the outer surface of the main cylinder 174t1, the communication hole 174b is opened at the rear end side of the step portion 174s to communicate with the insertion hole 174h1. Then, an annular filter surrounding portion 174d protrudes outward in the radial direction to surround the communication hole 174b. A disc-shaped filter member 180 is disposed inside the filter surrounding portion 174d to block the communication hole 174b. Further, a disc-shaped cover 182 is attached to the top portion of the ring of the filter surrounding portion 174d to be spaced from the filter member 180. The filter member 180 has air permeability and hydrophobic properties, and is formed of PTFE to be a continuous porous structure having continuous micro air holes.

Accordingly, a reference gas (external air) is introduced from the filter member 180 to the air passage hole of the gas detection element 22 via the communication hole 174b, the insertion hole 174h1, the air passage hole 23a, and the inward of the sensor terminal 24.

On the other hand, in the outer surface of the main cylinder 174t1, a lead wire surrounding portion 174g is formed at the opposite side of the filter surrounding portion 174d when seen from the axis O to protrude outward in the radial direction and is formed at the same level as the upper surface 174f. The lead wire surrounding portion 174g is formed in a rectangular parallelepiped shape, and the rear end side surface (that is, the upper surface 174f) of the lead wire surrounding portion 174g is provided with a lead wire communication groove 174w so as to allow the lead wire 153 to be in a connection state along the radial direction of the main cylinder 174t1.

Further, the cover portion 172 is substantially formed in a disc shape of which the dimension is equal to the outer diameter of the main cylinder 174t1, and is connected to the main cylinder 174t1 so that the leading end side surface 172f is evenly aligned to the upper surface 174f. Further, in the cover portion 172, a cover side lead wire surrounding portion 172g protrudes outward in the radial direction from a position facing the lead wire surrounding portion 174g with the contact surface between the leading end side surface 172f and the upper surface 174f interposed therebetween. Further, the leading end side surface of the cover side lead wire surrounding portion 172g is provided with a lead wire communication groove 172w.

Then, when the leading end side surface 172f of the cover portion 172 and the upper surface 174f of the main cylinder 174t1 are connected while facing each other, the lead wire 153 extending from the rear end of the cap terminal 151 via the lead wire rubber seal 162 is drawn to the outside of the cap body 170 via each of the lead wire communication grooves 172w and 174w.

The lead wire 153 is formed by coating a core wire with a coating material. The leading end of the core of the lead wire 153 is crimped by the core crimping portion 151b of the cap terminal 151 so as to be electrically connected to the sensor connection portion 151a. For this reason, the output signal may be transmitted from the inner electrode 29 of the gas detection element 22 of the gas sensor 21 to an external device (for example, an engine control unit (ECU)) via the lead wire 153.

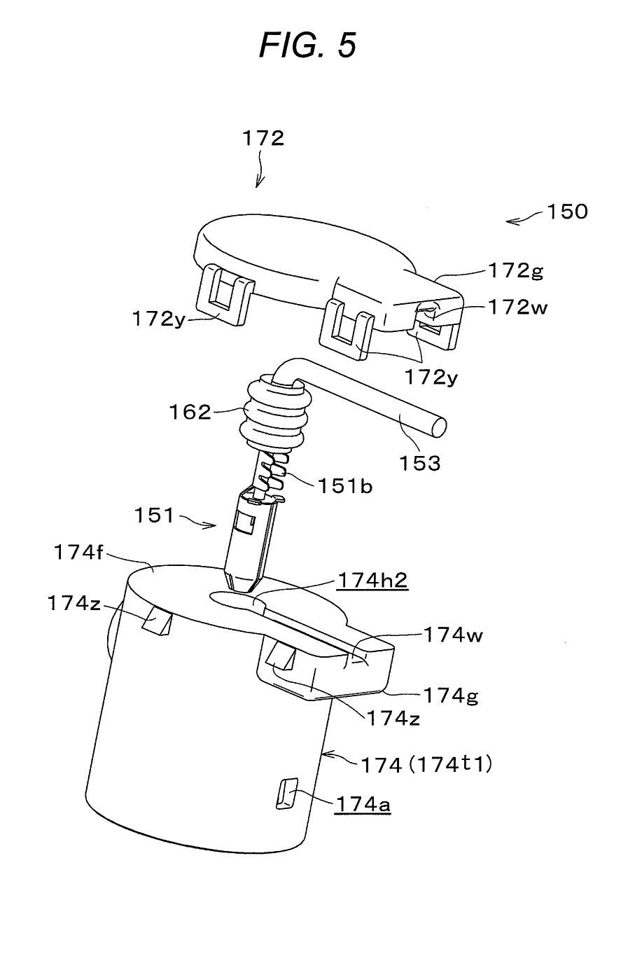

Next, an example of a method of manufacturing the sensor cap 150 will be described by reference to FIGS. 5 to 7. First, the assembling operation between the main cap body portion 174 and the cover portion 172 will be described by reference to FIG. 5. The leading end of the lead wire 153 is first crimped to the core crimping portion 151b of the rear end of the cap terminal 151 via the lead wire seal rubber 162. Then, the cap terminal 151 is inserted into the cap terminal hole 174h2 of the main cylinder 174t1 (refer to FIG. 3) of the main cap body portion 174, and the lead wire 153 is bent at the rear end side of the lead wire seal rubber 162 in the transverse direction substantially perpendicular to the axial direction O. Then, the lead wire rubber seal 162 protruding from the cap terminal hole 174h2 and the lead wire 153 bent in the transverse direction are pressed by the cover portion 172. At this time, as described below, since a press-insertion piece protrudes into the leading end side surface 172f of the cover portion 172, the cap terminal 151 and the lead wire seal rubber 162 are pressed into the cap terminal hole 174h2.

Further, plural (four in the embodiment) U-shaped locker parts 172y are disposed at the outside of the cover portion 172 so as to extend in axial direction O, and the center of the locker part 172y is opened in a rectangular shape. On the other hand, plural (four in the embodiment) protrusions 174z are disposed at the rear end side of the main cylinder 174t1 to protrude outward in the radial direction from the positions respectively corresponding to the locker parts 172y. Then, when the upper surface 174f of the main cylinder 174t1 is blocked by the cover portion 172, each protrusion 174z passes over a corresponding locker part 172y to engage with the opening at the center of the locker part 172y, and the cover portion 172 is fixed to the main cap body portion 174.

When the cap terminal 151 and the lead wire seal rubber 162 are pressed into the cap terminal hole 174h2 by (the press-insertion piece of) the cover portion 172, as described above, the cap terminal 151 is fixed to the inner cylinder 174t2 (refer to FIG. 3) at the position where the locking portion 174p is fitted to the separation prevention hole 151c. Further, since the lead wire rubber seal 162 is formed in a cylindrical shape, and the outer diameter thereof is slightly larger than the inner diameter of the rear end side of the cap terminal hole 174h2, the lead wire seal rubber 162 comes into close contact with the surface of the cap terminal hole 174h2 so as to obtain a waterproof assembly.

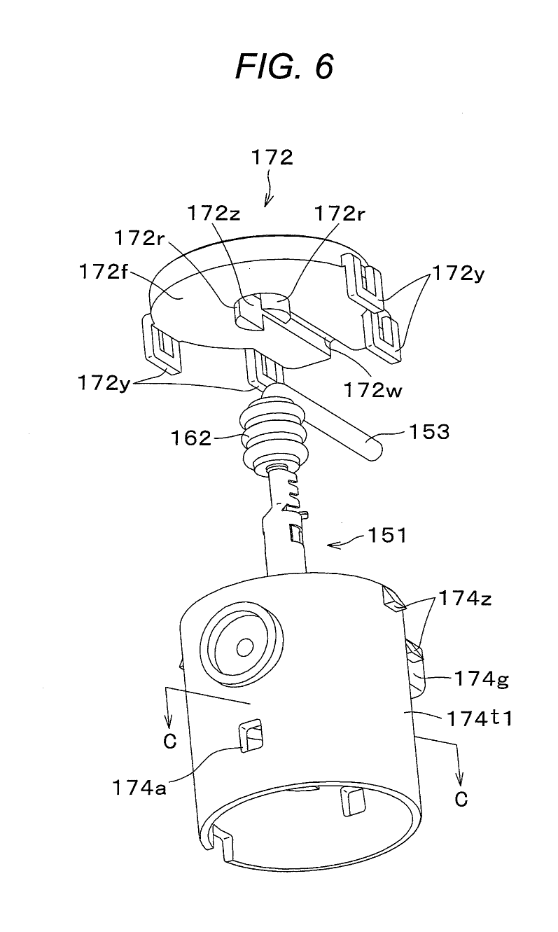

FIG. 6 illustrates a structure of the leading end side surface 172f of the cover portion 172. As described above, the lead wire rubber seal 162 is connected to the rear end of the cap terminal 151, and the lead wire 153 extends to the rear end of the lead wire rubber seal 162. In this case, since the lead wire 153 is connected to the lead wire rubber seal 162, an exclusive jig or process of pressing the lead wire rubber seal 162 into the cap terminal hole 174h2 (refer to FIG. 3) is needed.

Therefore, the lead wire communication groove 172w is provided in the leading end side surface 172f of the cover portion 172. Also, a press-insertion piece 172r is provided at the outer periphery of a central groove 172z of the cover portion 172 as the terminal end of the lead wire communication groove 172w so that the press-insertion piece 172r protrudes toward the leading end. The press-insertion piece 172r is provided along the inside of the outer periphery of the central groove 172z, and its cross-section perpendicular to the axial direction O is formed in a half-moon shape. Further, the press-insertion piece 172r is disposed at two facing positions on the outer periphery of the central groove 172z to pass over the lead wire 153 without interfering therewith, and to be perpendicular to the extension direction of the lead wire communication groove 172w. The outer diameter of the central groove 172z is substantially equal to the diameter of the rear end of the cap terminal hole 174h2, and the interval between the press-insertion pieces 172r is slightly wider than the outer diameter of the lead wire 153.

Accordingly, when the upper surface 174f of the main cylinder 174t1 is blocked by the cover portion 172, each press-insertion piece 172r passes over the lead wire 153 to be inserted into the cap terminal hole 174h2 to come into contact with the lead wire rubber seal 162. In this manner, the lead wire rubber seal 162 is pressed into the cap terminal hole 174h2. For this reason, an exclusive jig or a process of pressing the lead wire rubber seal 162 into the cap terminal hole is not needed, and the lead wire seal rubber 162 may be inserted into the cap terminal hole at the time of attaching the cover portion 172, whereby the productivity is enhanced. Further, since the length of the press-insertion piece 172r in the axial direction O is constant, the press-insertion depth of the lead wire rubber seal 162 may be constantly maintained, and the lead wire rubber seal 162 may be disposed at a predetermined position of the cap terminal hole 174h2.

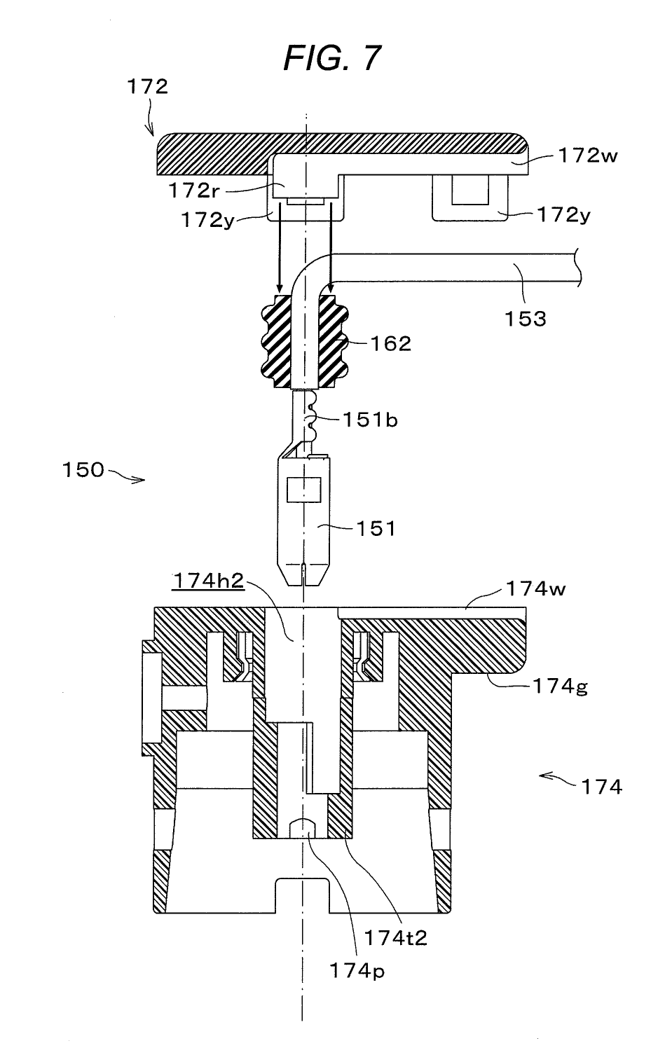

FIG. 7 is a cross-sectional view taken along the line C-C of FIG. 6. The length of the press-insertion piece 172r is substantially equal to the outer diameter of the lead wire rubber seal 162, and the lead wire rubber seal 162 may be sufficiently press-inserted. Further, the inner diameter of the cap terminal hole 174h2 is large at the rear end side, and a step portion is formed at the leading end side to decrease the diameter. Accordingly, the rear end side of the cap terminal hole 174h2 comes into close contact with the lead wire rubber seal 162, and the inner diameter of the leading end side is substantially equal to the outer diameter of (the sensor connection portion 151a of) the cap terminal 151, thereby reliably holding the cap terminal 151.

Then, as shown in FIG. 4, the cap body seal 160 and the seal stopper 176 are fitted into the insertion hole 174h1 of the main cap body portion 174 to hold the cap body seal 160. Further, at this procedure, originally the cap terminal 151, the lead wire seal 162, the lead wire 153, and the like are disposed in the main cap body portion 174, but this arrangement is not shown in FIG. 4. In this manner, the sensor cap 150 is completely assembled.

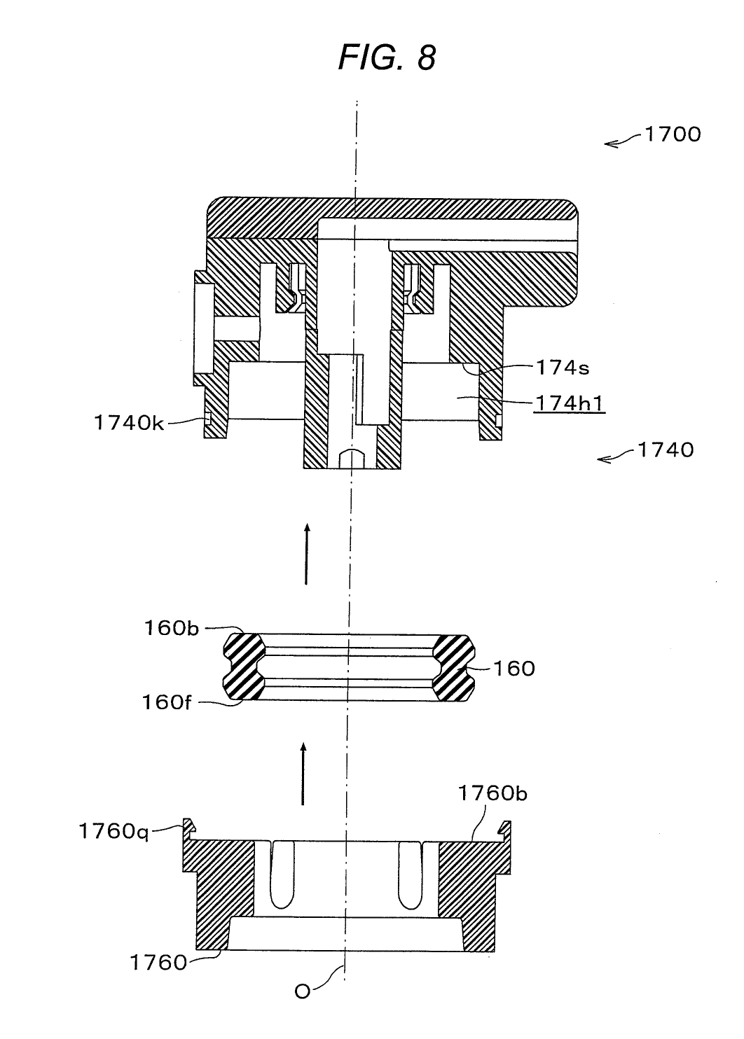

Next, the gas sensor unit according to the second embodiment will be described by referring to FIG. 8. Since the gas sensor unit according to the second embodiment has the same configuration as the gas sensor unit 200 according to the first embodiment except for the features of a main cap body portion 1740 and a seal stopper 1760, the description of the same configuration will not be repeated.

In the second embodiment, the length of the main cap body portion 1740 in the axial direction O is up to the rear edge of the rectangular hole 174a of the main cap body portion 174 of the first embodiment. On the other hand, the outer surface of the seal stopper 1760 is flat since there is no protrusion 176p, and the outer diameter of the seal stopper 1760 is set to be equal to the outer diameter of the main cap body portion 174 of the first embodiment. That is, in the second embodiment, a cap body 1700 is formed by coaxially coupling the seal stopper 1760 to the leading end of the main cap body portion 1740, and the length of the cap body 1700 in the axial direction O is the sum of the lengths of the main cap body portion 1740 and the seal stopper 1760 in the axial direction O.

Then, plural concave portions 1740k are formed at the leading end of the outer surface of the main cap body portion 1740 to be depressed inward in the radial direction. On the other hand, plural elastic pieces extend backward from the rear end of the outer surface of the seal stopper 1760, and a hook-shaped engagement claw 1760q is formed at the rear end of each elastic piece to protrude inward in the radial direction.

Accordingly, when the seal stopper 1760 is coaxially coupled to the leading end of the main cap body portion 1740, the engagement claw 1760q is pressed by the outer surface of the main cap body portion 1740 to be bent outward in the radial direction. Further, the engagement claw 1760q engages the concave portion 1740k at the position facing the concave portion 1740k, and the bending of the engagement claw 1760q is restored, thereby generating a clicking sensation. For this reason, since the sensation of mounting the seal stopper 1760 is obtained, the seal stopper 1760 may be reliably coupled to the main cap body portion 1740.

In this manner, the cap body seal 160 is held between the step portion 174s of the main cap body portion 1740 and the rear end side surface (leading end seal pressing portion) 1760b of the seal stopper 1760.

In the gas sensor unit according to the second embodiment, the cap body 1700 (the main cap body portion 1740 and the seal stopper 1760) is formed of a resin, and the cap body seal 160 held therebetween is formed of an elastic member such as rubber. Accordingly, the product cost may be reduced as compared with the case where the entire cap body 1700 is formed of a rubber member, or where a large rubber member is disposed to entirely cover a gap between the cap body 1700 and the gas sensor 21.

Further, both the rear end side and the leading end side of the cap body seal 160 may be held by the rear end seal pressing portion and the leading end seal pressing portion, so as to reliably hold the cap body seal 160.

Furthermore, even when thermal degradation occurs in the cap body seal 160 under practical use, the cap body seal 160 may be easily exchanged.

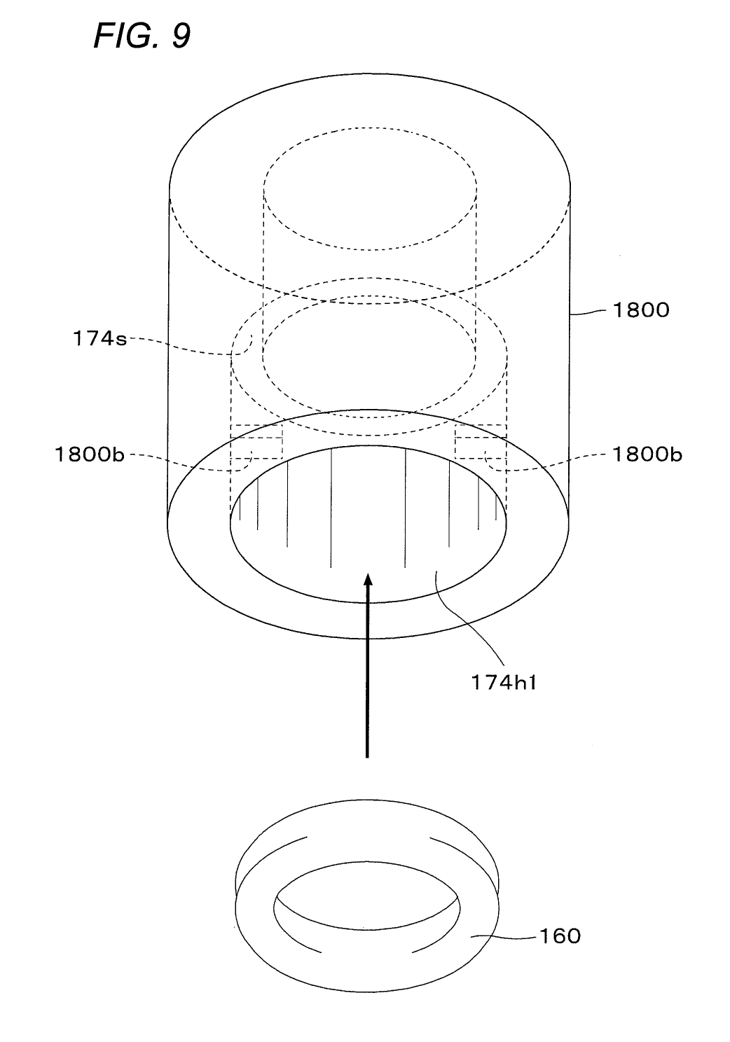

Next, the gas sensor unit according to a third embodiment will be described by reference to FIG. 9. In the gas sensor unit according to the third embodiment, a cap body 1800 is integrally formed without forming the main cap body portion and the seal stopper as separate components. However, since the other configurations are the same as those of the gas sensor unit 200 according to the first embodiment, their description will not be repeated. Further, the cover portion 172 separated from the cap body 1800 may be provided at the rear end side of the cap body 1800.

The third embodiment is the same as the first embodiment in that the cap body seal 160 is inserted into the insertion hole 174h1 to support the step portion 174s provided in the insertion hole 174h1 and the rear end side surface of the cap body seal 160. On the other hand, plural (two in this example) elastic pieces 1800b are provided at the leading end side of the step portion 174s in the insertion hole 174h1 to protrude inward in the radial direction. The elastic pieces 1800b are respectively provided at the positions facing each other in the radial direction of the insertion hole 174h1, and the interval between the step portion 174s and the elastic piece 1800b in the axial direction O is slightly larger than the thickness of the cap body seal 160.

Then, when the cap body seal 160 is compacted to an oval shape and inserted from the insertion hole 174h1 to the step portion 174s without contacting the elastic piece 1800b, the cap body seal 160 is restored to a round shape, and the leading end side surface of the cap body seal 160 comes into contact with the rear end side surface of the elastic piece 1800b. In this manner, the cap body seal 160 is held between the step portion 174s and the elastic piece 1800b. Further, the elastic piece 1800b corresponds to the “leading end seal pressing portion” of the claimed invention. At this time, the elastic pieces 1800b need not be respectively disposed at positions facing each other in the radial direction of the insertion hole 174h1, as long as the cap body seal 160 is held. For example, an odd number of the elastic pieces 1800b may be disposed at the same interval in the circumferential direction.

In the gas sensor unit according to the third embodiment, the cap body 1800 is formed of a resin, and the cap body seal 160 held between the step portion (rear end seal pressing portion) 174s and the elastic piece (leading end seal pressing portion) 1800b may be formed of an elastic member such as rubber. Accordingly, the product cost may be reduced as compared with the case where the entire cap body 1800 is formed of a rubber member, or where a large rubber member is disposed to entirely cover a gap between the cap body 1700 and the gas sensor 21. Further, both the rear end side and the leading end side of the cap body seal 160 may be held by the rear end seal pressing portion and the leading end seal pressing portion, so as to reliably hold the cap body seal 160. Furthermore, even when thermal degradation occurs in the cap body seal 160 under the practical use, the cap body seal 160 may be easily exchanged.

Further, the invention is not limited to the above-described embodiments, and needless to say, may be modified into various forms. For example, the gas sensor 21 is not limited to an oxygen sensor. Further, the shapes of the rear end seal pressing portion and the leading end seal pressing portion of the cap body are not limited. The shapes of the main cap body portion and the seal stopper and the combination method therebetween are not limited. Further, a concave portion may be provided in the inner surface of the insertion hole of the cap body to be continuous in the circumferential direction, and the concave portion may receive the cap body seal.

Furthermore, the sensor terminal may be of a male type, and the cap terminal may be of a female type.

It should further be apparent to those skilled in the art that various changes in form and detail of the invention as shown and described above may be made. It is intended that such changes be included within the spirit and scope of the claims appended hereto.

This application claims priority from Japanese Patent Application No. 2010-099493, filed on Apr. 23, 2010, and from Japanese Patent Application No. 2010-286994, filed on Dec. 24, 2010, the disclosures of which are incorporated herein by reference in their entirety.

What is claimed is:

1. A gas sensor unit comprising:
a gas sensor including:
a gas detection element which extends in an axial direction and has a cylinder shape with a bottom in which a leading end side is closed,
a sensor terminal which is inserted into a round hole of the gas detection element to be electrically connected to an inner electrode formed at an inner surface of the gas detection element and having a rear end protruding from a rear end side of the gas detection element, and
a cylindrical surrounding body which surrounds a periphery of the rear end of the sensor terminal; and
a sensor cap including:
a cap terminal which is electrically connected to the sensor terminal, and
a cap body which has an insertion hole surrounding at least a part of the rear end of the surrounding body, the cap body surrounding a periphery of the cap terminal,
wherein the cap body is formed of a resin, and a cap body seal surrounding the surrounding body is fitted into the insertion hole, and
wherein the cap body includes:
a rear end seal pressing portion that contacts with a rear end side surface of the cap body seal; and
a leading end seal pressing portion that contacts a leading end side surface of the cap body seal,
wherein the cap body seal is hold by the rear end seal pressing portion and the leading end seal pressing portion of the cap body.

2. The gas sensor unit according to claim 1,
wherein the cap body includes:
a main cap body portion having the rear end seal pressing portion; and
a seal stopper fitting into the insertion hole and having the leading end seal pressing portion, the main cap body portion and the seal stopper being provided as separate components, and
wherein the seal stopper is coupled to the main cap body portion at a leading end side of the cap body seal so as to hold the cap body seal.

3. The gas sensor unit according to claim 2,
wherein a concave portion or an open portion is provided at a leading end side of the main cap body portion, and
wherein the seal stopper has an outward engagement portion protruding outward in a radial direction to engage the concave portion or the open portion, and the seal stopper is fitted to an inside of the main cap body portion.

4. The gas sensor unit according to claim 2,
wherein an outer surface of the main cap body portion has a concave portion or an open portion, and
wherein the seal stopper has an inward engagement portion protruding inward in a radial direction to engage with the concave portion or the open portion, and the seal stopper is fitted to an outside of the main cap body portion.

5. The gas sensor unit according to claim 1, further comprising:
a metal shell which supports the gas detection element; and
a metallic outer cylinder which surrounds an outside of the surrounding body,
wherein the outer cylinder is fixed to the metal shell.

6. The gas sensor unit according to claim 5,
wherein the outer cylinder is crimped and fixed to the metal shell.

7. The gas sensor unit according to claim 1,
wherein at least a part of the cap body seal is located at a rear end side of the sensor cap rather than at a center thereof when viewed from the axial direction.
